(19) United States
(12) Plant Patent Application Publication
(10) Pub. No.: US 2003/0196233 P1
(43) Pub. Date: Oct. 16, 2003

(54) Spray rose plant named 'JACrewhi'

(76) Inventor: Keith W. Zary, Somis, CA (US)

Correspondence Address: BURNS, DOANE, SWECKER & MATHIS, L.L.P., P.O. Box 1404, Alexandria, VA, 22313-1404, US

(21) Appl. No.: 10/121,670

(22) Filed: Apr. 15, 2002

(51) Int. Cl. A01H 5/00

(52) U.S. Cl. PLT/143

(57) ABSTRACT

A new and distinct cultivar of the Spray Class is provided that abundantly forms in clusters attractive double irregularly striped pale pink and cream blossoms. The blossoms possess no fragrance. An erect growth habit is exhibited. The foliage is semi-glossy and dark green and contrasts well with the striped blossoms. The disease resistance is very good. The plant is well suited for the production of cut floral sprays under greenhouse growing conditions.

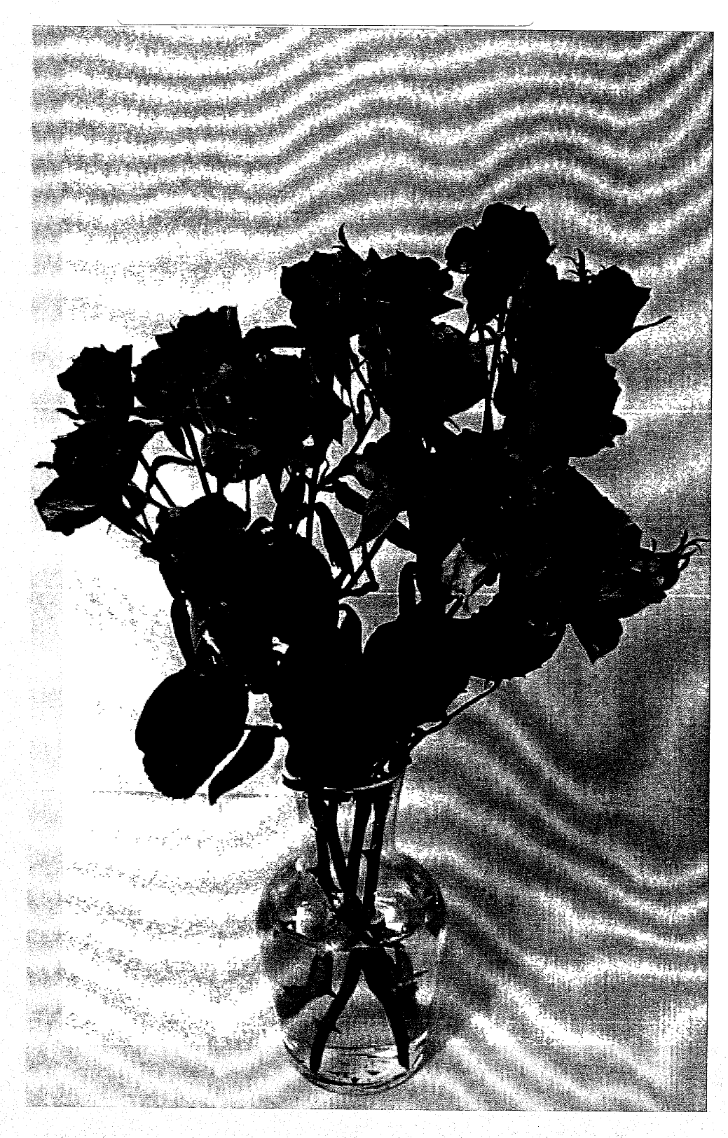

### BOTANICAL/COMMERCIAL CLASSIFICATION

Rosa hybrida/Spray Rose

### VARIETAL DENOMINATION

cv. ‘JACrewhi’

### SUMMARY OF THE INVENTION

The present invention is a new and distinct cultivar of Spray Rose plant, botanically known as Rosa hybrida, and hereafter is referred to by the cultivar name ‘JACrewhi’.

The new cultivar was created at Somis, Calif., U.S.A., by artificial pollination wherein two parents were crossed which previously had been studied in the hope that they would contribute the desired characteristics. The female parent (i.e., the seed parent) of the new cultivar was the ‘JACeve’ cultivar (non-patented in the United States) and the male parent (i.e., the pollen parent) was the ‘JAComail’ cultivar (non-patented in the United States). The parentage of the new cultivar can be summarized as follows:

‘JACeve’×‘JAComail’.

The seeds resulting from the above pollination were sown and small plants were obtained which were physically and biologically different from each other. Selective study resulted in the identification of a single plant of the new variety.

It was found that the Spray Rose plant of the present invention possesses the following combination of characteristics:

(a) an erect growth habit,

(b) forms in clusters attractive double irregularly striped pale pink and cream blossoms that possess no fragrance,

(c) forms sturdy stems,

(d) forms attractive dark green leaves having a semi-glossy aspect, and

(e) exhibits very good disease resistance.

The striped inflorescence coloration contrasts nicely with the dark green foliage.

The new cultivar well meets the needs of the horticultural industry. It is particularly well-suited for use in the production of cut floral sprays under greenhouse growing conditions.

The new cultivar has been found to undergo asexual propagation at Somis, Calif., U.S.A., by budding and by the rooting of cuttings. Asexual propagation by these routes has shown that the characteristics of the new cultivar are firmly fixed and are strictly transmissible from one generation to another.

### BRIEF DESCRIPTION OF THE PHOTOGRAPHY

The accompanying photograph shows as nearly true as is reasonably possible to make the same in a color illustration of this character, typical specimens of the new cultivar. The attractive double striped pale pink and cream blossoms, dark green foliage, and stems are illustrated while present in a transparent vase. The plants were grown under standard greenhouse growing conditions.

### DETAILED DESCRIPTION

The description is based upon the observation of plants of the new cultivar growing in a greenhouse at Somis, Calif., U.S.A. The growing conditions approximate those commonly utilized for the commercial production of cut roses.

Growth Habit:

Erect.

Foliage:

Attractive dark green and semi-glossy on the upper surface. The leaf serration is simple and regular.

Stems:

Sturdy and commonly possess rather large thorns (as illustrated).

Inflorescence:

The blossoms are double and commonly are borne in sprays. The blossom coloration is attractive irregularly striped pale pink and cream. Some dark purple-red coloration sometimes appears at the petal borders. Such appearance is influenced by the stage of the growing season. The blossoms possess no fragrance.

Disease Resistance:

Very good.

Propagation:

Budding and the rooting of cuttings.

Usage:

Production of cut floral sprays under greenhouse growing conditions.

I claim:

1. A new and distinct cultivar of Rosa hybrida plant having the following combination of characteristics:
(a) an erect growth habit,
(b) forms in clusters attractive double irregularly striped pale pink and cream blossoms that possess no fragrance,
(c) forms sturdy stems,
(d) forms attractive dark green leaves having a semi-glossy aspect, and
(e) exhibits very good disease resistance;
substantially as illustrated and described.
